(19) United States
(12) Patent Application Publication
(10) Pub. No.: US 2023/0138669 A1
(43) Pub. Date: May 4, 2023

(54) Peripherally Inserted Central Catheter System Including a Stylet with Lubrication

(71) Applicant: Bard Access Systems, Inc., Salt Lake City, UT (US)

(72) Inventor: Yiping Ma, Layton, UT (US)

(21) Appl. No.: 17/977,836

(22) Filed: Oct. 31, 2022

Related U.S. Application Data
(60) Provisional application No. 63/274,468, filed on Nov. 1, 2021.

(51) Int. Cl. A61M 25/01; A61M 25/00

(52) U.S. Cl. CPC A61M 25/0102; A61M 25/0045; A61M 25/0082; A61M 25/005; A61M 2025/0047

(57) ABSTRACT

A catheter assembly including catheter and a stylet inserted within a central lumen of the catheter. The catheter may be peripherally inserted central catheter including a hub coupled to an elongate body defining a central lumen with one or more extension legs coupled to the hub. The stylet includes a stylet tube having a plurality of magnets together with a lubricant disposed within a lumen of the stylet tube. The lubricant enables sliding displacement of the magnets within the lumen during a bending event of the stylet. A wire may also be disposed within the lumen of stylet tube and the wire may wrapped around each magnet. An attachment member of the stylet may be coupled with a connector of the catheter. An electrode may be coupled to the wire.

### PRIORITY

This application claims the benefit of priority to U.S. Provisional Application No. 63/274,468, filed Nov. 1, 2021, which is incorporated by reference in its entirety into this application.

### BACKGROUND

Inserting a peripherally inserted central catheter (“PICC”) into the vasculature of the patient may involve a stylet being placed inside a lumen of the PICC to give the PICC stabilization during insertion of a PICC-stylet assembly. However, the tip of the PICC can encounter obstructions during placement, leading to buckling or a bending event of the tips of the stylet and the PICC. The bending event can lead to breaking off of the tips of the stylet and the PICC, wherein breaking off of one or more of the tips is considered a serious failure of the PICC. It would be beneficial to the patient and the clinician to have a PICC and a stylet that are not prone to a bending event, allowing the clinician to properly place the PICC without fear of PICC or stylet failure. Disclosed herein are a system, apparatus and method of manufacturing that address the foregoing.

### SUMMARY

Disclosed herein is catheter assembly, that according to some embodiments, includes a catheter defining a catheter proximal end and a catheter distal end, where the catheter defines a central lumen extending between the catheter distal end and a catheter connector at the catheter proximal end. The catheter assembly further includes aa stylet inserted into the central lumen where the stylet includes (i) a stylet tube defining a tube lumen extending along the stylet tube between a stylet proximal end and a stylet distal end, (ii) a plurality of magnets disposed within the tube lumen, and (iii) a lubricant disposed between one or more of the plurality of magnets and an inner surface of the stylet tube such that the lubricant enables sliding motion the one or more of the plurality of magnets with respect to the stylet tube.

In some embodiments, the catheter is a peripherally inserted central catheter. In some embodiments, the catheter includes two or more extension legs, each extension leg defining an extension leg lumen in fluid communication with the central lumen.

In some embodiments, the stylet tube includes a polyamide material.

In some embodiments, the lubricant is disposed between the plurality of magnets and an inner surface of the stylet tube. In some embodiments, the lubricant extends between adjacent magnets of the plurality of magnets.

In some embodiments, the plurality of magnets are disposed along a magnetic region of the stylet, where the magnetic region extends proximally away from the stylet distal end. In some embodiments, the lubricant is disposed along the magnetic region. In some embodiments, the lubricant is disposed along a portion of the stylet tube extending proximally away from the magnetic region.

In some embodiments, each of the magnets is cylindrically shaped, and the plurality of magnets are arranged end-to-end along the magnetic region. In some embodiments, each of the magnets includes a parylene coating.

In some embodiments, the stylet includes a stainless steel wire disposed within the tube lumen, where the wire extends longitudinally along at least the magnetic region. In some embodiments, the wire is wrapped at least partially around each magnet.

In some embodiments, the stylet includes one or more sensors configured to detect electrical signals and, in some embodiments, the one or more sensors includes an electrode disposed at the distal end of the stylet, where the electrode is electrically coupled with the wire.

In some embodiments, the lubricant is water soluble, and in some embodiments, the lubricant includes a biologic agent.

In some embodiments, the stylet includes an attachment member configured to couple with a catheter connector. In some embodiments, the attachment member includes a male Luer taper configured engage a female Luer taper of the catheter connector.

In some embodiments, a first distance between the attachment member and the stylet distal end is substantially equal to a second distance between the catheter connector and the catheter distal end such that, when the attachment member is coupled with the connector, the stylet distal end is disposed adjacent the catheter distal end.

Also disclosed herein is a stylet configured for insertion within a catheter that, according to some embodiments, includes (i) a stylet tube defining a tube lumen extending along the stylet tube between a stylet proximal end and a stylet distal end, (ii) a plurality of magnets disposed within the tube lumen, and (iii) a lubricant disposed between one or more of the plurality of magnets and an inner surface of the stylet tube such that the lubricant enables sliding motion the one or more of the plurality of magnets with respect to the stylet tube.

In some embodiments of the stylet, the stylet tube includes a polyamide material.

In some embodiments of the stylet, the lubricant is disposed between the plurality of magnets and an inner surface of the stylet tube and in some embodiments of the stylet, the lubricant is disposed between adjacent magnets of the plurality of magnets.

In some embodiments of the stylet, the plurality of magnets are disposed along a magnetic region of the stylet, where the magnetic region extends proximally away from the stylet distal end. In some embodiments of the stylet, the lubricant is disposed along the magnetic region.

In some embodiments, the stylet includes a stainless steel wire disposed within the tube lumen, where the stainless steel wire extends longitudinally along at least the magnetic region. In some embodiments of the stylet, the stainless steel wire is wrapped at least partially around each magnet.

In some embodiments, the stylet includes one or more sensors configured to detect electrical signals. In some embodiments of the stylet, the one or more sensors includes an electrode disposed at the distal end of the stylet, the electrode electrically coupled with the wire.

In some embodiments, the stylet includes an attachment member configured to couple with the catheter connector.

Also disclosed herein is a method of manufacturing a catheter assembly, where the method includes assembling a stylet, and where assembling a stylet includes (i) dispensing a lubricant within a lumen of a stylet tube, where the lubricant disposed adjacent a distal end of the stylet tube; and (ii) inserting a plurality of magnets within the lumen of the stylet tube such that the lubricant covers each of the plurality of magnets. The method further includes inserting the stylet into a central lumen of a catheter.

In some embodiments of the method, 33. The method according to claim 21, wherein the catheter includes a peripherally inserted central catheter having more than one extension leg, each extension leg including an extension leg lumen in fluid communication with the central lumen.

In some embodiments of the method, assembling the stylet further includes wrapping the each of the plurality of magnets with a wire, and inserting the wire into the lumen of the stylet tube.

In some embodiments of the method, assembling the stylet further includes (i) coupling an electrode to the stylet tube at the distal end, (ii) connecting the wire to the electrode, and (iii) extending the wire proximally beyond a proximal end of the stylet tube.

In some embodiments, the method further includes positioning the stylet with respect to the catheter such that the distal end of the stylet tube is disposed adjacent a distal end of the catheter.

In some embodiments of the method, assembling the stylet further includes threading an attachment member onto the stylet tube and securing the attachment member to the stylet tube, and the method further includes coupling the attachment member to a connector of the catheter.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a peripherally inserted central catheter system including a peripherally inserted central catheter and a stylet, in accordance with some embodiments;

FIGS. 2A-2B illustrate a cross-sectional side view of the distal end of the stylet of FIG. 1 in a bending event, in accordance with some embodiments;

FIG. 3A illustrates a detailed cross-sectional side view of the stylet, in accordance with some embodiments;

FIG. 3B illustrates a cross sectional end view of the distal end of the stylet, in accordance with some embodiments;

FIGS. 4A-4B illustrate a cross-sectional view of the distal end of the stylet including the lubricant in a bending event, in accordance with some embodiments; and

FIG. 5 illustrates a flow chart of an exemplary method of manufacturing the catheter system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

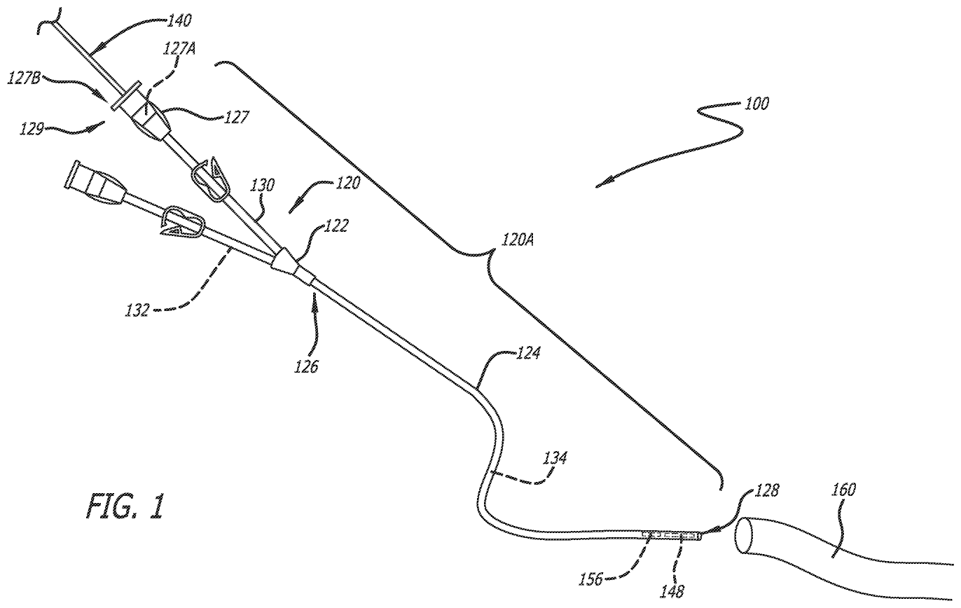

FIG. 1 illustrates a perspective view of a catheter assembly (“assembly”) 100 including a catheter 120, coupled with a stylet 140, in accordance with some embodiments. In some embodiments, the catheter 120 includes a peripherally inserted central catheter (PICC). The catheter 120 defines a central lumen 134 extending along an elongate body 124, and the stylet 140 is inserted into the central lumen 134, when the stylet 140 is coupled with the catheter 120. The catheter 120 includes a catheter connector 127 at the proximal end 129 of the catheter 120. The catheter connector 127 is positioned a distance 120A away from a distal end 128 of the catheter 120. In some embodiments, the catheter connector 127 may include a female Luer taper 127A and/or external Luer locking threads 127B. In some embodiments, the catheter 120 includes one or more extension legs 130 coupled to a hub 122, where the one or more extension legs 130 extend proximally away from the hub 122, and where each extension leg defines an extension leg lumen 132 therein, and where each extension leg lumen 132 is in fluid communication with the central lumen 134. However, it will be appreciated that in some embodiments, the assembly 100 may be designed to include PICCs having two or more lumens, which are considered to fall within the scope of this disclosure.

In some embodiments, the stylet 140 may be inserted through the one or more extension leg lumens 132 into the central lumen 134 to provide rigidity to the catheter 120 so as to enable insertion of the catheter 120 into the vasculature of the patient. The stylet 140 may include a plurality of magnets 148, where the plurality of magnets 148 are configured to generate a magnetic field so that the stylet 140 is detectable and/or trackable via a magnetic tracking medical device. In some embodiments, the stylet 140 may include one or more sensors 156 (e.g., electrodes or coils) configured to detect one or more electrical signals and provide the electrical signals to a computing device. In an embodiment, the one or more sensors 156 may be configured to detect electrical signals emanating from the patient, such as an ECG signal emanating from the heart of the patient when as the stylet 140 is inserted into the vasculature of the patient.

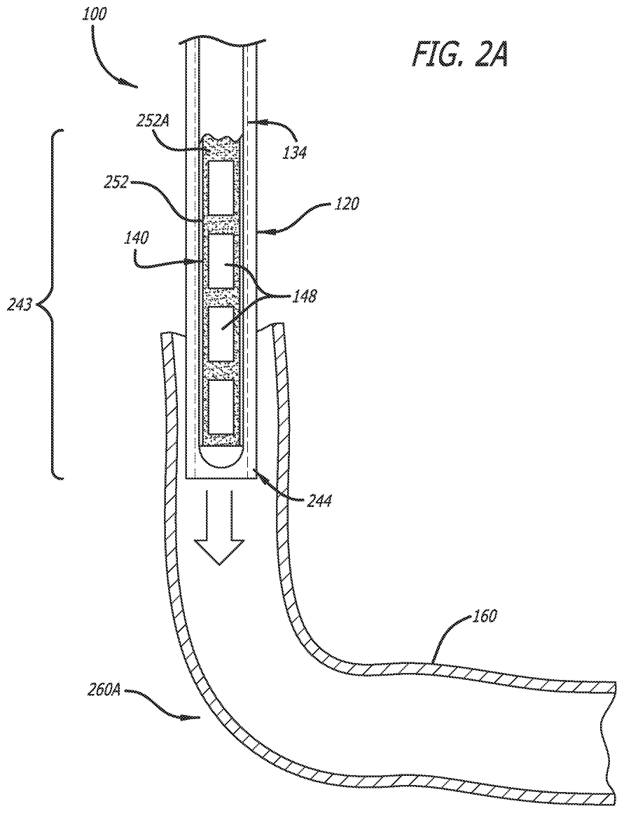

FIGS. 2A-2B illustrates a cross-sectional side view a distal portion of the stylet 140 and the catheter 120 traversing a blood vessel 160 including a resistance 260A of the blood vessel 160, such as a curve, for example. The stylet 140 includes a stylet tube or tubing 252 extending along a length of the stylet 140, where the tubing 252 defines a lumen 252A and the plurality of magnets 148 is disposed within the lumen 252A. As illustrated in FIG. 2A, in some embodiments, the stylet 140 is inserted into the central lumen 134 of the catheter 120. As the catheter assembly 100 is advanced along the lumen of the blood vessel 160, the catheter assembly 100 may encounter the resistance 260A (e.g., the illustrated curve, an increase or decrease in blood vessel diameter, or the like). The catheter assembly 100 may be forced through the resistance 260A, to continue advancement along the blood vessel 160. In some instances, the distal end 244 may engage the resistance 260A causing the stylet 140 undergo a bending event, as illustrated in FIG. 2B. The bending event includes the a distal portion of the stylet 140, such as the magnetic region 243, for example, undergoing excess strain due to the short gauge length (or short radius of curvature) of tubing 252 along the magnetic region 243. The excess strain may cause rupturing (or tearing) of the tubing 252, where the rupturing may cause a portion of the stylet 140, such as a portion adjacent the distal end 244 of the stylet 140 to separate from the stylet 140 and so as to become a free particle within the blood vessel 160.

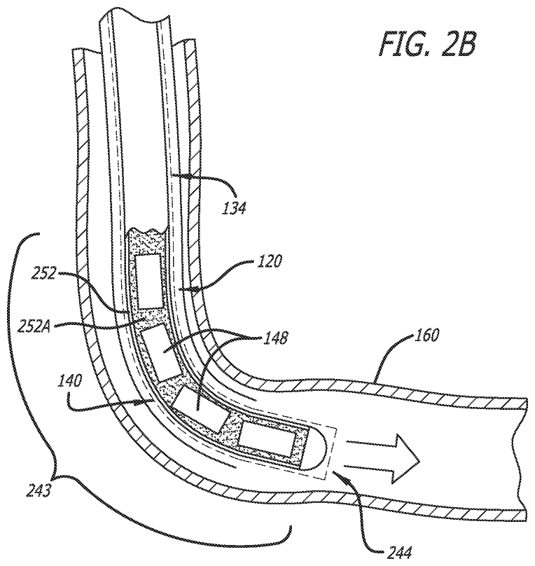

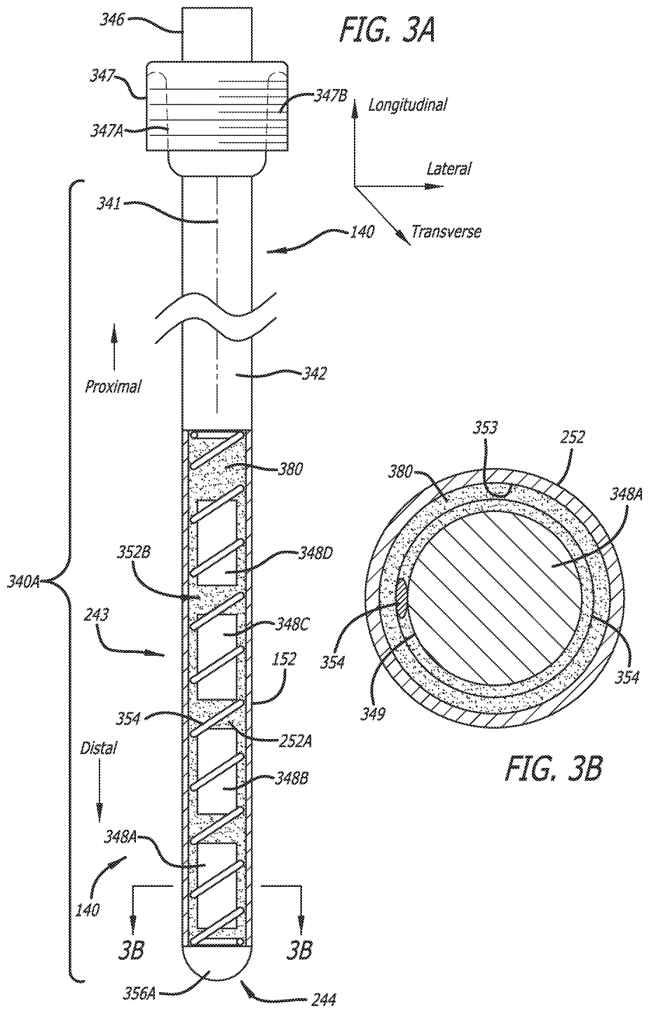

FIG. 3A illustrates a cross-sectional side view of the stylet 140 including a distal end 244, in accordance with some embodiments. In some embodiments, the stylet 140 includes a stylet body 342 having a proximal end 346 and the distal end 244. In some embodiments, the stylet body 342 may include an elongate body defining a central longitudinal axis 341. In some embodiments, the stylet 140 may include an attachment member 347 configured to detachably couple the stylet 140 to the catheter 120 via the catheter connector 127. In some embodiments, the attachment member 347 may be configured to position the stylet 140 including the distal end 244 in an optimal position within the catheter 120. In some embodiments, a distance 340A between the attachment member 347 and the distal end 244 may be substantially equal to the distance 120A (see FIG. 1) such that when the stylet 140 is coupled with the catheter 120, the distal end 244 of the stylet 140 is disposed adjacent the distal end 128 of the catheter 120.

In some embodiments, the attachment member 347 may include components consistent with a male Luer lock connector. For example, the attachment member 347 may include a male Luer taper 347A configured to engage a female Luer taper 127A (see FIG. 1) of the of the catheter connector 127. Similarly, the attachment member 347 may include internal Luer locking threads 347B configured to engage external Luer locking threads 127B (see FIG. 1) of the catheter connector 127.

In some embodiments, the stylet body 342 may include a singular piece body or may have a multi-piece body. In some embodiments, the entire stylet body 342 may be rigid or portions of the stylet body 342 may be rigid and some portions of the stylet body 342 may be flexible. For example, a portion of the stylet body 342 extending distally away from the proximal end 346 may be more rigid than a portion of the stylet body 342 extending proximally away the distal end 244. In some embodiments, the tubing 252 may include polyimide tubing, PVC, silicon, or the like.

As discussed above, the stylet 140 includes magnetic region 243 extending proximally away from the distal end 244. The magnetic region 243 includes the plurality of magnets 148 (See FIGS. 1-2B). In the illustrated embodiment, the plurality of magnets 148 includes four magnets 348A-348D. In other embodiments, the plurality of magnets 148 may include more or less than four magnets. The magnets 348A-348D are configured to generate a magnetic field detectable by a magnetic tracking medical device. In some embodiments, the magnets 348A-348D may extend to the distal end 244 of the stylet 140. In some embodiments, the magnets 348A-348D may be aligned consecutively along the central longitudinal axis 341. For example, a first magnet 348A may be disposed adjacent the distal end 244 and a second magnet 348B may be disposed proximal the first magnet 348A. In some embodiments, the tubing 252 may be sheathed over the plurality of magnets 148. In some embodiments, the magnets 348A-348D may be cylindrically shaped and may be arranged end-to-end.

In some embodiments, the stylet 140 may include a wire 354 extending along the stylet 140. In some embodiments, the wire 354 may extend only along the magnetic region 243. In other embodiments, the wire 354 extend along an entire length of the stylet 140. The wire 354 may be wrapped (i.e., coiled) at least partially around each of the magnets 348A-348D. In some embodiments, the wire 354 may be wrapped (i.e., coiled) at least one full revolution around each of the magnets 348A-348D. As shown, the wire 354 may be disposed within the lumen 252A of the tubing 252. In some embodiments, the wire 354 may be formed of stainless steel. In some embodiments, the wire 354 may be configured to secure the magnets 348A-348D to the stylet 140 and/or limit longitudinal displacement of the magnets 348A-348D with the lumen 252A. In some embodiments, the wire 354 may define a separation space 352B between adjacent magnets, such as the magnets 348A, 348B, for example.

In some embodiments, the wire 354 may be configured to propagate electrical signals along the stylet 140. In some embodiments, proximal portion of the wire 354 may extend beyond a proximal end 346 of the stylet 140 (or more specifically the tubing 252), so that the wire 354 may be connected to a medical device configured to process electrical signals, such as an ECG device, for example. In some embodiments, the one or more sensors 156 may include an electrode 356A disposed at the distal end 244 of the stylet 140 and the wire 354 may be electrically connected to the electrode 356A.

In some embodiments, the stylet 140 may include a lubricant 380, where the lubricant 380 is generally configured to enable longitudinal sliding displacement of one or more of the magnets 348A-348D with respect to the tubing 252. In some embodiments, the lubricant 380 may be disposed between a first magnet 348A and a second magnet 348B, the second magnet 348B and a third magnet 348C, and the third magnet 348C and a fourth magnet 348D. In some embodiments, the lubricant 380 may extend from the distal end 244 of the stylet 140 to a proximal end of the magnetic region 243. By enabling sliding displacement of the one or more of the magnets 348A-348D with respect to the tubing 252, the lubricant 380 enables one or more of the magnets 348A-348D to longitudinally displace during the bending event, thereby decreasing strain on the tubing 252 during the bending event to prevent, inhibit, or otherwise reduce the likelihood of a rupture of the tubing 252.

FIG. 3B illustrates a cross sectional view of the stylet 140, in accordance with some embodiments. In an embodiment, the stylet body 342 may include a multi-piece body extending to the distal end 244. In some embodiments, each of the magnets 348A-348D may define toroidal cross-section. In some embodiments, adjacent magnets may be magnetically attracted to each other. In some embodiments, the lubricant 380 may define a layer of lubrication disposed between an outer surface 349 one or more of the magnets 348A-348D and an inner surface 353 of the tubing 252. In some embodiments, the wire 354 may be disposed within the layer of the lubricant 380. In some embodiments, the lubricant 380 may be disposed within the space 352B between adjacent magnets, and the force of the insertion of the stylet 140 into the catheter 120 may cause the lubricant 380 to spread along the outer surface 349 of the magnets 348A-348D and the inner surface 153 of the tubing 252. The lubricant 380 may be a liquid or a semi-solid (e.g., a gel). In some embodiments, the lubricant 380 may be water soluble. In some embodiments, the lubricant 380 may include biological agent, such as a drug, for example.

In some embodiments, the lubricant 380 may include a composition that includes one or more of silicone oil, glycerin, hydroxyethyl cellulose, hypromellose, propylene glycol, hydroxypropyl methylcellulose, or the like. In some embodiments, the lubricant 380 is applied to the stylet 140 during the manufacturing process. In some embodiments, the lubricant 380 may include a formulation that transitions between an inert state and an active state upon movement, friction, temperature or the like. In some embodiments, the lubricant 380 may be (i) applied to the stylet 140 in the inert state and then (ii) transitioned to an active state before or during insertion of the stylet 140. In an embodiment, the lubricant 380 may be formed of a thin solid film that liquefies upon being heated above a defined temperature, allowing the lubricant 380 to flow between the magnets 348A-348D and the tubing 252. In an embodiment, the defined temperature may be greater than 95° F.

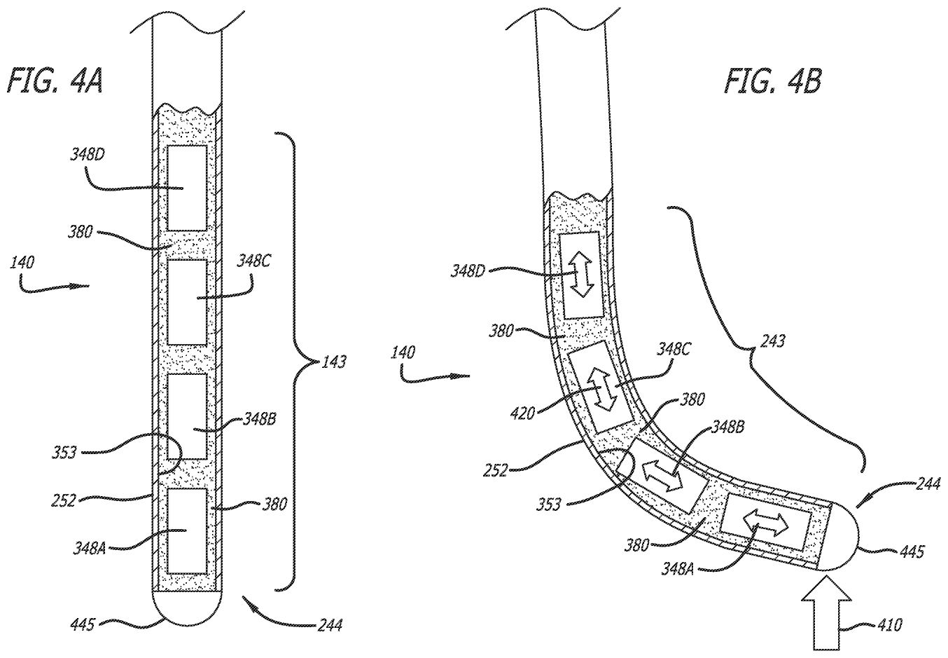

FIGS. 4A-4B illustrate a cross-sectional view of the distal end 244 of the stylet 140 including the magnets 348A-348D disposed along the magnetic region 243 and the lubricant 380 disposed within the lumen 252A, in accordance with some embodiments. FIG. 4A illustrates the magnetic region 243 disposed in a straight shape and FIG. 4B illustrates the magnetic region 243 disposed in a curved shape resulting from a force 410 applied to a distal tip 445 of the stylet 140, the force 410 causing a bending event. As shown by arrows 420, independent bidirectional sliding displacement of the each of the magnets 348A-348D with respect to the tube 252 may occur at-will during a bending event as a result of the reduced friction provided by the lubricant 380. As such, the lubricant 380 inhibits sliding contact of the magnets 348A-348D with the inside surface 153 from gouging or otherwise damaging the tube 252. Similarly, the at-will independent bidirectional sliding displacement of the each of the magnets 348A-348D may enable the tube 252 to bend at a larger radius of curvature, thus reducing strain in the tubing 252, thereby further inhibiting damage to the tube 252.

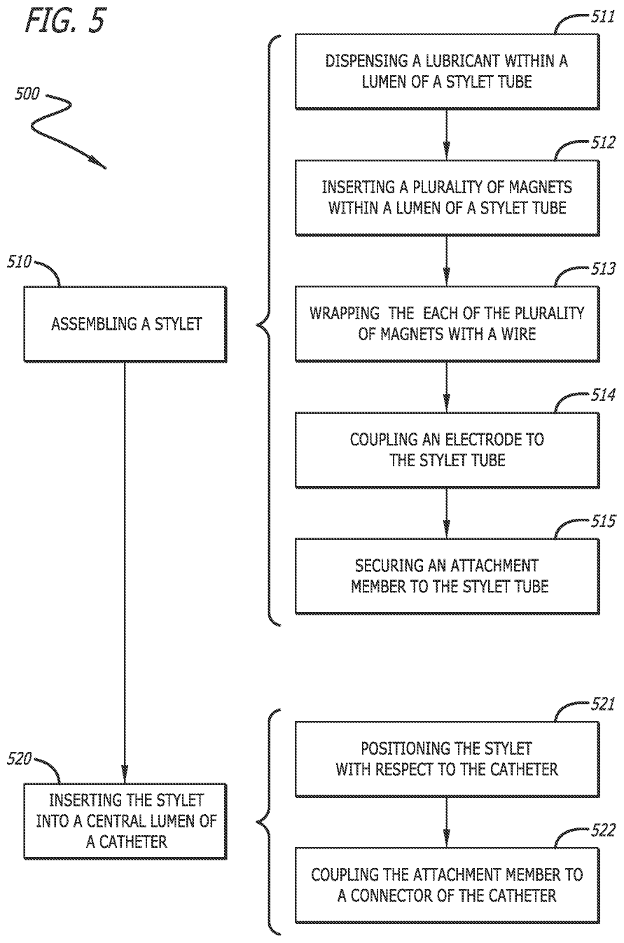

FIG. 5 illustrates a flow chart of an exemplary method of manufacturing of the catheter assembly, in accordance with some embodiments. The method 500 may include all or any subset of the following steps, actions, or processes. In some embodiments, the method 500 may include assembling a stylet (block 510).

Assembling the stylet may include dispensing a lubricant within a lumen of a stylet tube (block 511). Dispensing the lubricant may include locating the lubricant adjacent the distal end of the stylet tube, such as along the magnetic region, for example.

Assembling the stylet may also include inserting the plurality of magnets within the lumen of the stylet tube (block 512). Inserting the plurality of magnets may include locating the magnets adjacent the distal end of the stylet tube, such as along the magnetic region, for example. Dispensing the lubricant and/or inserting the magnets may include covering the magnets with the lubricant.

Assembling the stylet may also include wrapping the each of the plurality of magnets with a wire (block 513) and inserting the wire into the lumen of the stylet tube (block 514).

Assembling the stylet may also include coupling an electrode to the stylet tube (block 515), where the electrode is coupled to the stylet tube at the distal end. Coupling the electrode may also include connecting the wire to the electrode, and extending the wire proximally beyond a proximal end of the stylet tube.

Assembling the stylet may also include securing an attachment member to the stylet tube (block 516) to the stylet tube. In some embodiments, securing an attachment member includes threading the attachment member onto the stylet tube.

The method 500 may also include inserting the stylet into a central lumen of a catheter (block 520). The catheter may include a peripherally inserted central catheter having more than one extension leg, where each extension includes an extension leg lumen in fluid communication with the central lumen.

Inserting the stylet into the central lumen of the catheter may also include positioning the stylet with respect to the catheter (block 521) such that the distal end of the stylet tube is disposed adjacent a distal end of the catheter.

Inserting the stylet into the central lumen of the catheter may also include coupling the attachment member to a connector of the catheter (block 522).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter assembly, comprising:
a catheter defining a catheter proximal end and a catheter distal end, the catheter defining a central lumen extending between the catheter distal end and a catheter connector at the catheter proximal end;
a stylet inserted into the central lumen, comprising:
a stylet tube defining a tube lumen extending along the stylet tube between a stylet proximal end and a stylet distal end,
a plurality of magnets disposed within the tube lumen, and
a lubricant disposed between one or more of the plurality of magnets and an inner surface of the stylet tube such that the lubricant enables sliding motion the one or more of the plurality of magnets with respect to the stylet tube.

2. The catheter assembly according to claim 1, wherein the catheter is a peripherally inserted central catheter.

3. The catheter assembly according to claim 2, wherein the catheter includes two or more extension legs, each extension leg defining an extension leg lumen in fluid communication with the central lumen.

4. The catheter assembly according to claim 1, wherein the stylet tube includes a polyamide material.

5. The catheter assembly according to claim 1, wherein the lubricant is disposed between the plurality of magnets and an inner surface of the stylet tube.

6. The catheter assembly according to claim 1, wherein the lubricant extends between adjacent magnets of the plurality of magnets.

7. The catheter assembly according to claim 1, wherein each of the plurality of magnets includes a parylene coating.

8. The catheter assembly according to claim 1, wherein the plurality of magnets are disposed along a magnetic region of the stylet, the magnetic region extending proximally away from the stylet distal end.

9. The catheter assembly according to claim 8, wherein the lubricant is disposed along the magnetic region.

10. The catheter assembly according to claim 8, wherein the lubricant is disposed along a portion of the stylet tube extending proximally away from the magnetic portion.

11. The catheter assembly according to claim 8, wherein:
each of the magnets is cylindrically shaped, and
the plurality of magnets are arranged end-to-end along the magnetic region.

12. The catheter assembly according to claim 8, wherein;
the stylet includes a stainless steel wire disposed within the tube lumen, and
the stainless steel wire extends longitudinally along at least the magnetic region.

13. The catheter assembly according to claim 12, wherein the stainless steel wire is wrapped at least partially around each magnet.

14. The catheter assembly according to claim 1, wherein the stylet includes one or more sensors configured to detect electrical signals.

15. The catheter assembly according to claim 14, wherein the one or more sensors includes an electrode disposed at the distal end of the stylet, the electrode electrically coupled with the wire.

16. The catheter assembly according to claim 1, wherein the lubricant is water soluble.

17. The catheter assembly according to claim 1, wherein the lubricant includes a biologic agent.

18. The catheter assembly according to claim 1, wherein the stylet includes an attachment member configured to couple with the catheter connector.

19. The catheter assembly according to claim 18, wherein the attachment member includes a male Luer taper configured to engage a female Luer taper of the catheter connector.

20. The catheter assembly according to claim 18, wherein a first distance between the attachment member and the stylet distal end is substantially equal to a second distance between the catheter connector and the catheter distal end such that, when the attachment member is coupled with the connector, the stylet distal end is disposed adjacent the catheter distal end.

21-37. (canceled)
